(19) United States
(12) Patent Application Publication
(10) Pub. No.: US 2004/0067936 A1
(43) Pub. Date: Apr. 8, 2004

(54) Process for preparation of hydrates of olanzapine and their conversion into crystalline forms of olanzapine

(76) Inventors: Buchi Reddy Reguri, Hyderabad (IN); Ramesh Chakka, Hyderabad (IN); Robert Koprowski, Upper Saddle River, NJ (US)

Correspondence Address: LADAS & PARRY, 26 WEST 61ST STREET, NEW YORK, NY, 10023, US

(21) Appl. No.: 10/363,436

(22) PCT Filed: Mar. 7, 2001
(86) PCT No.: PCT/US01/07258

(30) Foreign Application Priority Data
Aug. 31, 2000 (IN) 709/MAS/2000
Aug. 31, 2000 (IN) 711/MAS/2000

(51) Int. Cl. A61K 31/551; C07D 487/12

(52) U.S. Cl. 514/220; 540/558

(57) ABSTRACT

The present invention relates to a method for the preparation of hydrates of 2-methyl-4-(4-methyl-1-piperazinyl)-10H-thieno [2,3-b] [1,5] benzodiazepine (hereinafter referred to as Olanzapine). The present invention also relates to a process for conversion of these hydrates into a pure crystalline form of olanzapine referred to as form-I. The present invention also relates to a method of converting Olanzapine Form-2 to Form-1.

The present invention relates to a method for the preparation of hydrates of 2-methyl-4-(4-methyl-1-piperazinyl)-10H-thieno [2,3-b][1,5] benzodiazepine (hereinafter referred to as Olanzapine). The present invention also relates to a process for conversion of these hydrates into a pure crystalline form of olanzapine referred to as form-I. The present invention also relates to a method of converting Olanzapine Form-2 to Form-1.

This invention more particularly relates to the preparation of hydrates of olanzapine and their conversion into crystalline form of Olanzapine Form-1 through recrystallization from a solvent. Olanzapine is represented by the following structure.

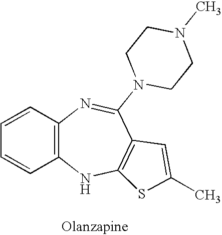

Olanzapine is useful for treating psychotic patients and mild anxiety states. Preparation of Olanzapine and its acid salts, having pharmaceutical properties particularly in the treatment of disorders of the central nervous system has been discussed in U.S. Pat. No. 5,229,382.

U.S. Pat. No. 5,229,382 does not refer to any specific polymorphic crystalline form of Olanzapine. European patent specification No. 733635A1 claims Form-2 of Olanzapine. The process under this patent describes preparation of Form-2 from ethyl acetate. This patent also designated the product obtained according to the process described in U.S. Pat. No. 5,229,382 as Form-1.

Furthermore, EP 733635A1 discloses the d values for Form-1 and Form-2 from their X-ray Diffractograms. The values are:

| d value | d value |
|---|---|
| Form-1 | Form-2 |
| 9.94 | 10.26 |
| 8.55 | 8.57 |
| 8.24 | 7.47 |
| 6.88 | 7.12 |
| 6.37 | 6.14 |
| 6.24 | 6.07 |
| 5.58 | 5.48 |
| 5.30 | 5.21 |
| 4.98 | 5.12 |
| 4.83 | 4.98 |
| 4.72 | 4.76 |
| 4.62 | 4.71 |
| 4.53 | 4.47 |
| 4.46 | 4.33 |
| 4.29 | 4.22 |
| 4.23 | 4.14 |
| 4.08 | 3.98 |
| 3.82 | 3.72 |
| 3.74 | 3.56 |
| 3.69 | 3.53 |
| 3.58 | 3.38 |
| 3.50 | 3.25 |
| 3.33 | 3.12 |
| 3.28 | 3.08 |
| 3.21 | 3.06 |
| 3.11 | 3.01 |
| 3.05 | 2.87 |
| 2.94 | 2.81 |
| 2.81 | 2.72 |
| 2.75 | 2.64 |
| 2.65 | 2.60 |
| 2.63 | |
| 2.59 | |

It is noteworthy to mention that EP 0 831 098 A2 discloses the preparation of a series of dihydrates of olanzapine namely Dihydrate B, Dihydrate D and Dihydrate B. The d values from the X-ray Diffractograms for these forms are listed in EP 0 831 098 A2.

We conducted experiments to obtain Olanzapine Form I by recrystallization of olanzapine from acetonitrile using the process described in Example 1, sub example 4 of U.S. Pat. No. 5,229,382. The process is described herein for reference: A mixture of 4-amino-2-methyl-10H-thieno-[2,3-b] [1,5]benzodiazepine HCl (100 g), N-methyl piperizine (350 ml), DMSO (465 ml) and toluene (465 ml) was heated to reflux. The reaction mass was maintained at reflux for 19 hours and then cooled to 50° C. and water was added. The reaction mass was cooled to 0-10° C. and stirred at the same temperature for 6 hours. The crude Olanzapine separated was filtered and dried in oven to a constant weight (76.5 g). The crude compound was added to acetonitrile (750 ml) at boiling temperature. The mixture was boiled for further 5 minutes. The mixture was filtered to remove the undissolved solid. The filtrate was treated with carbon and filtered. The filtrate was distilled to a minimum volume, cooled to 0-5° C. and maintained at the same temperature for 1.0 hour and filtered. The compound was dried to a constant weight in an oven (51.6 g).

The polymorphic form obtained from these experiments was characterized for its X-ray Powder Diffraction on Rigaku D/Max 2200. As clearly observed, the d values for this product (FIG. 1) matched with those of Olanzapine Form-2 claimed in EP 733635A1. It is therefore inferred that the recrystallization of Olanzapine in acetonitrile produces Form-2 and not Form-1.

Accordingly, the present invention provides a novel method for preparation of hydrates of olanzapine, which are different from those reported in the literature. These hydrates are named Olanzapine monohydrate-I and Olanzapine dihydrate-I for convenience.

Accordingly, the present invention also provides a novel method for preparation of Olanzapine Form-1 by recrystallization of olanzapine or its hydrates in dichloromethane. The present invention also provides a novel method for converting Olanzapine Form-2 to Olanzapine Form-1

According to the present invention the process for the preparation of olanzapine monohydrate-I comprises:

a) refluxing a mixture of 4-amino-2-methyl-10H-thieno-[2,3-b][1,5]benzodiazepine hydrochloride, N-methyl piperazine, dimethyl sulfoxide (DMSO) and toluene for 5 to 20 hours;

b) cooling the mixture to 20 to 90° C.;

c) adding water;

d) cooling the mixture to −5 to 25° C. and stirring for 2-10 hours;

e) filtering the mixture and washing with water; and

f) drying at 30 to 50° C. to a constant weight.

According to the present invention the process for the preparation of olanzapine dihydrate-I comprises:

a) refluxing a mixture of 4-amino-2-methyl-10H-thieno-[2,3-b][1,5]benzodiazepine hydrochloride, N-methyl piperazine, dimethyl sulfoxide (DMSO) and toluene for 5 to 20 hours;

b) cooling the mixture to 20 to 90° C.;

c) adding water;

d) cooling the mixture to −5 to 25° C. and stirring for 2-10 hours;

e) filtering the mixture and washing with water; and

f) drying at ambient temperature to a constant weight.

The preferred ratio of 4-amino-2-methyl-10H-thieno-[2,3-b] [1,5] benzodiazepine HCl, N-methyl piperizine, DMSO and toluene that can be used for preparation of the monohydrate and dihydrate are:

N-methyl piperizine (2.0-8.4 moles with respect to 1.0 mole of 4-Amino-2-methyl-10H-thieno-[2,3-b] [1,5]benzodiazepine HCl).

DMSO (2-8 times by volume with respect to 1.0 mole of 4-Amino-2-methyl-10H-thieno-[2,3-b] [1,5]benzodiazepine HCl).

Toluene (3-8 times by volume with respect to 1.0 mole of 4-Amino-2-methyl-10H-thieno-[2,3-b] [1,5]benzodiazepine HCl).

According to this invention, Olanzapine Form-I is prepared by heating to reflux a suspension of olanzapine or its hydrates in dichloromethane wherein the amount of dichloro-methane used is 4.5 to 13 volume/weight of Olanzapine to obtain a clear solution. The resultant solution is then treated with carbon followed by filtration. Upon completion of this step the filtrate is cooled to 0 to 5° C. and stirred at the same temperature for 60-90 minutes. The separated solid was filtered and washed with dichloromethane. The product obtained on drying in an oven at 60-70° C. to a constant weight is Form-1 of Olanzapine.

The process described in U.S. Pat. No. 5,229,382 was used to prepare olanzapine crude and the process described in EP 733 635 A1 was used to prepare olanzapine Form-2 for our studies. However, other methods may be used to prepare olanzapine crude and olanzapine Form-2 and any other methods that can be used to prepare olanzapine crude and olanzapine Form 2 can be used in the processes of this invention.

The following examples are provided for purposes of illustration and are not to be construed as limiting the scope of the invention.

### Preparation of Olanzapine Monohydrate-1

### EXAMPLE 1

A mixture of 4-amino-2-methyl-10H-thieno-[2,3-b][1,5]benzodiazepine hydrochloride (20 Kg), N-methyl piperazine (42 lit), dimethyl sulfoxide (40 lit) and toluene (95 lit) was heated to reflux. The reaction mass was maintained at reflux for 17 hours and 15 minutes and then cooled to 40-50° C. Water (95 lit) was added slowly at 40-50° C. The reaction mass was cooled to −0.6 to 1.2° C. and stirred at the same temperature for six hours. The Olanzapine crude that separated was filtered and washed with water (10 lit). The product was dried at 30.5 to 3 1.8° C. for 10 hrs and 50 minutes. Yield: 20 Kg. A 20 gm sample from the above material after prolonged heating for an additional 72 hours gave the product with a moisture content of 5.22%.

### Preparation of Olanzapine Dihydrate-I

### EXAMPLE 2

A mixture of 4-amino-2-methyl-10H-thieno-[2,3-b][1,5]benzodiazepine hydrochloride (200 g), N-methyl piperazine (420 ml), dimethyl sulfoxide (200 ml) and toluene (940 ml) was heated to reflux. The reaction mass was maintained at reflux for 12 hours and then cooled to 40° C. Water (940 ml) was added slowly at 40-44° C. The reaction mass was cooled to 0-5° C. and stirred at the same temperature for five hours. The Olanzapine crude that separated was filtered and washed with water (100 ml). The solid obtained was dried atmospherically (25-35° C.) for 24 hours (Yield: 241 g).

### Preparation of Form-I

### EXAMPLE 3

Crude 2-methyl-4-(4-methyl-1-piperazinyl)-10H-thieno-[2,3-b][1,5] benzodiazepine (35.0 g) was suspended in dichloromethane (160.0 ml). The suspension was heated to reflux to obtain a clear solution. The resultant solution was then treated with carbon (3.5 g) followed by filtration. Upon completion of this step the filtrate was cooled to 0 to 5° C. and stirred at the same temperature for one hour. The separated solid was filtered and washed with chilled dichloromethane (10.0 ml). The product obtained on drying in oven at 65 to 70° C. to a constant weight gave Form-1 of Olanzapine (Yield 22.0 g).

### Conversion Of Form-2 To Form-1

### EXAMPLE 4

The stirred suspension of pure form-2 of 2-methyl-4-(4-methyl-1-piperazinyl)-10H-thieno-[2,3-b][1,5]benzodiazepine (20.0 g) in dichloromethane (90.0 ml) was heated to reflux to obtain a clear solution. The clear solution was filtered and the filtrate was then cooled to 3 to 5° C. and stirred at same temperature for one hour. The crystalline solid separated was filtered and washed with dichloromethane (4.0 ml). Subsequent drying at 60 to 70° C. to a constant weight yielded Olanzapine Form-1. (Yield: 12.7 g).

### Preparation of Form-1 from Monohydrate-I of Olanzapine

### EXAMPLE 5

Monohydrate-I of 2-methyl-4-(4-methyl-1-piperazinyl)-10H-thieno-[2,3-b][1,5] benzo-diazepine (25.0 g) prepared as per Example-1 was suspended in dichloromethane (325.0 ml). The suspension was heated to reflux to obtain a clear solution. The resultant solution was then treated with carbon (2.5 g) followed by filtration. Upon completion of this step the filtrate was distilled to a minimum volume and then cooled to 2 to 4° C. and stirred at the same temperature for 90 minutes. The product separated was filtered and washed with chilled dichloromethane (10 ml). The product obtained on drying in oven at 60 to 70° C. to a constant weight gave Form-1 of Olanzapine (Yield 16.5 g)

### Preparation of Form-1 From Dihydrate-I of Olanzapine

### EXAMPLE 6

Dihydrate-I of 2-methyl-4-(4-methyl-1-piperazinyl)-10H-thieno-[2,3-b][1,5] benzodiazepine (40.0 g) prepared as per Example-2 was suspended in dichloromethane (520.0 ml). The suspension was heated to reflux to obtain a clear solution. The resultant solution was then treated with carbon (4.0 g) followed by filtration. Upon completion of this step the filtrate was distilled to a minimum volume and the left over reaction mass was cooled to 0 to 2° C. and stirred at the same temperature for one hour. The separated solid was filtered and washed with dichloromethane (10.0 ml). The product obtained on drying in oven at 65 to 70° C. to a constant weight renders Form-1 of Olanzapine (Yield 26.0 g).

The aforementioned crystalline forms in examples 1 to 6 have been examined for their structural and analytical data viz., Powder X-Ray Diffraction, Differential Scanning Calorimetry, and Infrared Absorption Spectroscopy. The results obtained are discussed and the respective drawings attached (FIGS. 2-19).

The X-Ray Diffraction Pattern set out herein for examples 1 to 6 were obtained using Rigaku D/Max-2200 X-Ray Powder Diffractometer having a copper K radiation source of wavelength λ=1.54 A°. The samples were scanned between 3-45 degrees 2θ.

The d values for the monohydrate-1 in Example-1 are herewith enclosed (FIG. 2).

| d value | I/Iₒ |
|---|---|
| 10.0176 | 100 |
| 6.8995 | 7 |
| 6.3567 | 12 |
| 6.1714 | 11 |
| 4.8756 | 51 |
| 4.7262 | 22 |
| 4.5904 | 34 |
| 4.4937 | 7 |
| 4.4315 | 13 |
| 4.3414 | 10 |
| 4.1411 | 6 |
| 3.9174 | 9 |
| 3.8669 | 23 |
| 3.7857 | 26 |
| 3.6480 | 9 |
| 3.5701 | 15 |
| 3.4451 | 3 |
| 3.2500 | 4 |
| 3.2065 | 4 |
| 2.9646 | 5 |
| 2.8715 | 3 |
| 2.8572 | 2 |

The d values for the dihydrate-1 in Example-2 are herewith given (FIG. 5).

| d value | I/Iₒ |
|---|---|
| 9.9949 | 100 |
| 9.6887 | 7 |
| 7.0418 | 2 |
| 6.4117 | 2 |
| 6.2495 | 7 |
| 6.1205 | 6 |
| 5.4534 | 6 |
| 5.2358 | 2 |
| 4.8230 | 33 |
| 4.7162 | 9 |
| 4.5717 | 15 |
| 4.4847 | 6 |
| 4.3924 | 8 |
| 4.3080 | 4 |
| 4.2070 | 3 |
| 4.0735 | 3 |
| 3.9974 | 3 |
| 3.9242 | 9 |
| 3.8438 | 12 |
| 3.7699 | 9 |
| 3.7386 | 13 |
| 3.6837 | 3 |
| 3.6509 | 4 |
| 3.6072 | 5 |
| 3.5256 | 11 |
| 3.4242 | 2 |
| 3.1773 | 2 |
| 3.1207 | 2 |
| 2.9917 | 2 |
| 2.9569 | 3 |
| 2.8733 | 2 |
| 2.8483 | 2 |

The X-Ray Diffraction Pattern obtained for the products from examples 3 to 6 is identical with those reported in EP 733 635 A1.

### BRIEF DESCRIPTION OF DRAWINGS

FIG. 1 is a characteristic X-ray powder diffraction pattern of Form-2 obtained on recrystallization with acetonitrile (Vertical axis: Intensity (CPS); Horizontal axis: Two Theta (degrees)).

FIG. 2 is a characteristic X-ray powder diffraction pattern of Olanzapine monohydrate-I (Vertical axis: Intensity.(CPS); Horizontal axis: Two Theta (degrees)).

FIG. 3 is a characteristic infrared absorption spectrum in potassium bromide of Olanzapine monohydrate-I (Vertical axis, Tramission (%); Horizontal axis: Wave number (cm⁻¹)).

FIG. 4 is a characteristic of differential scanning calorimetry thermogram of Olanzapine monohydrate-I. (Vertical axis: mW; Horizontal axis: Temperature (° C.)).

FIG. 5 is a characteristic X-ray powder diffraction pattern of Olanzapine dihydrate-I (Vertical axis: Intensity (CPS); Horizontal axis: Two Theta (degrees)).

FIG. 6 is a characteristic infrared absorption spectrum in potassium bromide of Olanzapine dihydrate-I. (Vertical axis, Tramission (%); Horizontal axis: Wave number (cm⁻¹)).

FIG. 7 is a characteristic of differential scanning calorimetry thermogram of Olanzapine dihydrate-I. (Vertical axis: mW; Horizontal axis: Temperature (° C.)).

FIG. 8 is a characteristic X-ray powder diffraction pattern of Form-I produced by recrystallizing crude Olanzapine in dichloromethane. (Vertical axis: Intensity (CPS); Horizontal axis: Two Theta (degrees)).

FIG. 9 is a characteristic infrared absorption spectrum in potassium bromide of Form-1 produced by recrystallizing crude Olanzapine in dichloromethane. (Vertical axis, Tramission (%); Horizontal axis: Wave number (cm⁻¹)).

FIG. 10 is a characteristic of differential scanning calorimetry thermogram of Form-I produced by recrystallizing crude Olanzapine in dichloromethane. [Vertical axis: mW; Horizontal axis: Temperature (° C.))].

FIG. 11 is a characteristic X-ray powder diffraction pattern of Form-I obtained on conversion of Form-2 to Form-1 Olanzapine in dichloromethane (Vertical axis: Intensity (CPS); Horizontal axis: Two Theta (degrees)).

FIG. 12 is a characteristic infrared absorption spectrum in potassium bromide of Form-1 obtained on conversion of Form-2 to Form-1 Olanzapine in dichloromethane (Vertical axis, Tramission (%); Horizontal axis: Wave number (cm⁻¹))].

FIG. 13 is a characteristic of differential scanning calorimetry thermogram of Form-1 obtained on conversion of Form-2 to Form-1 Olanzapine in dichloromethane (Vertical axis: mW; Horizontal axis: Temperature (° C.))].

FIG. 14 is a characteristic X-ray powder diffraction pattern of Form-1 obtained on conversion of olanzapine monohydrate-I to Form-1 Olanzapine in dichloromethane (Vertical axis: Intensity (CPS); Horizontal axis: Two Theta (degrees)).

FIG. 15 is a characteristic infrared absorption spectrum in potassium bromide of Form-1 obtained on conversion of olanzapine monohydrate-I to Form-1 Olanzapine in dichloromethane (Vertical axis, Tramission (%); Horizontal axis: Wave number (cm⁻¹))

FIG. 16 is a characteristic of differential scanning calorimetry thermogram of Form-1 obtained on conversion of olanzapine monohydrate-I to Form-1 Olanzapine in dichloromethane. (Vertical axis: mW; Horizontal axis: Temperature (° C.)).

FIG. 17 is a characteristic X-ray powder diffraction pattern of Form-1 obtained on conversion of olanzapine dihydrate-I to Form-1 Olanzapine in dichloromethane (Vertical axis: Intensity (CPS); Horizontal axis: Two Theta (degrees)).

FIG. 18 is a characteristic infrared absorption spectrum in potassium bromide of Form-1 obtained on conversion of olanzapine dihydrate-I to Form-1 Olanzapine in dichloromethane. ([Vertical axis, Tramission (%); Horizontal axis: Wave number (cm⁻¹)]).

FIG. 19 is a characteristic of differential scanning calorimetry thermogram of Form-1 obtained on conversion of olanzapine dihydrate-I to Form-1 Olanzapine in dichloromethane. (Vertical axis: mW; Horizontal axis: Temperature (° C.)).

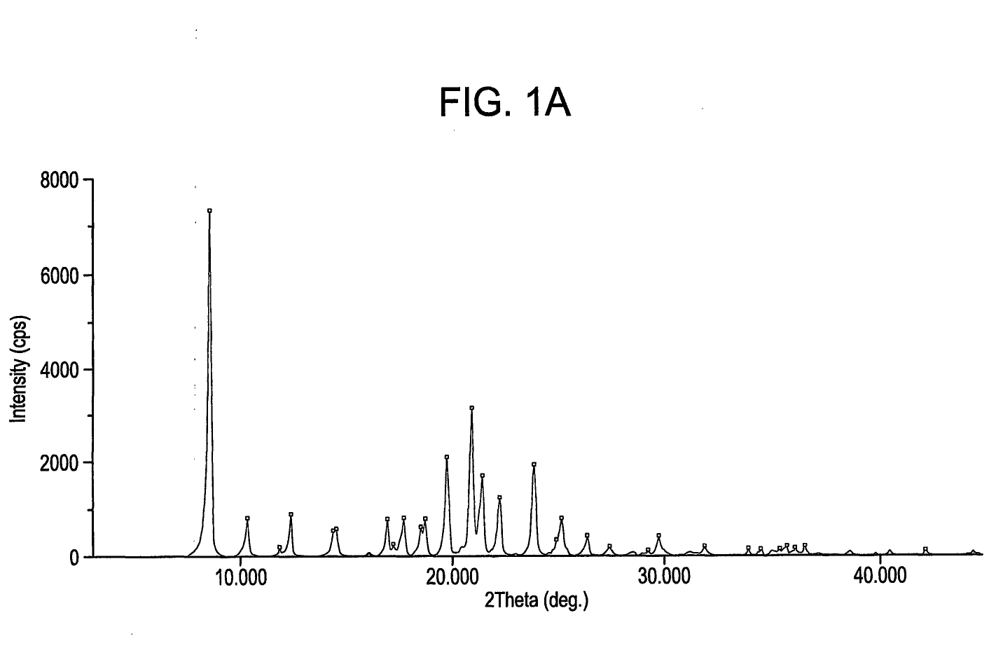

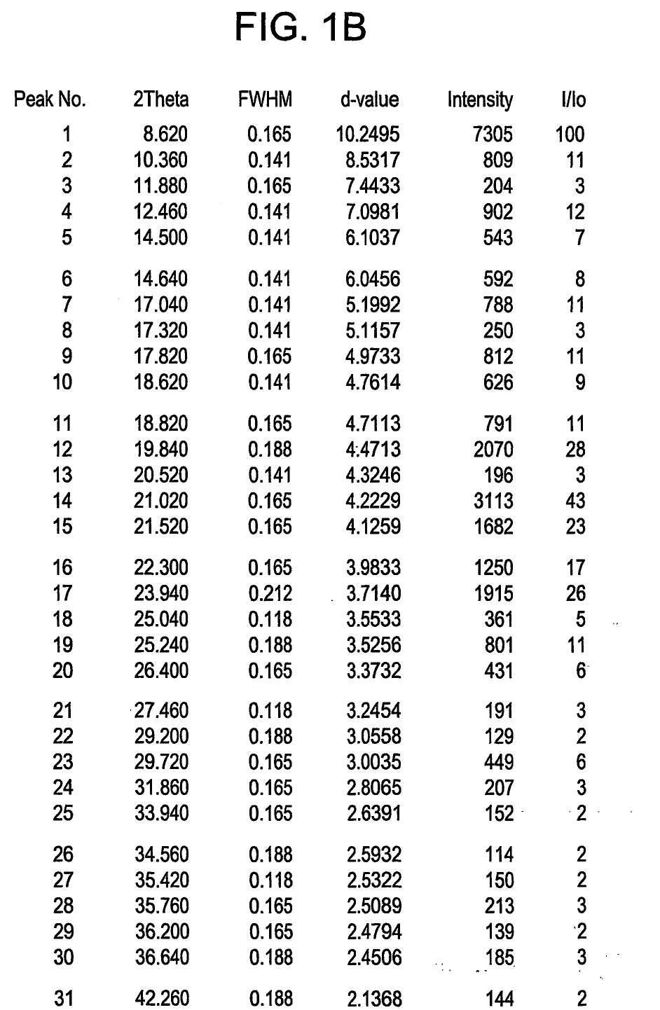

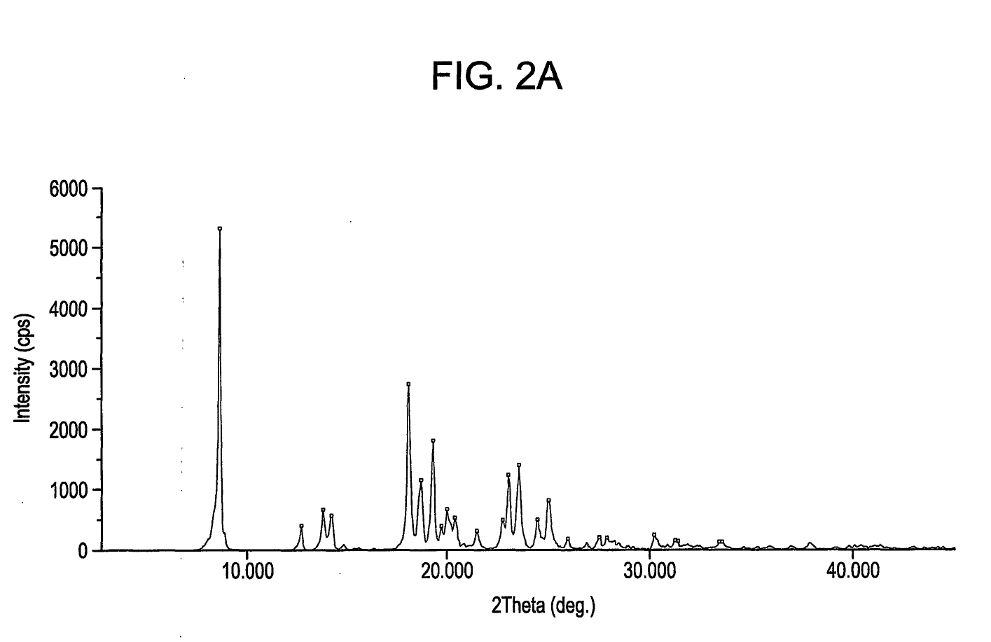

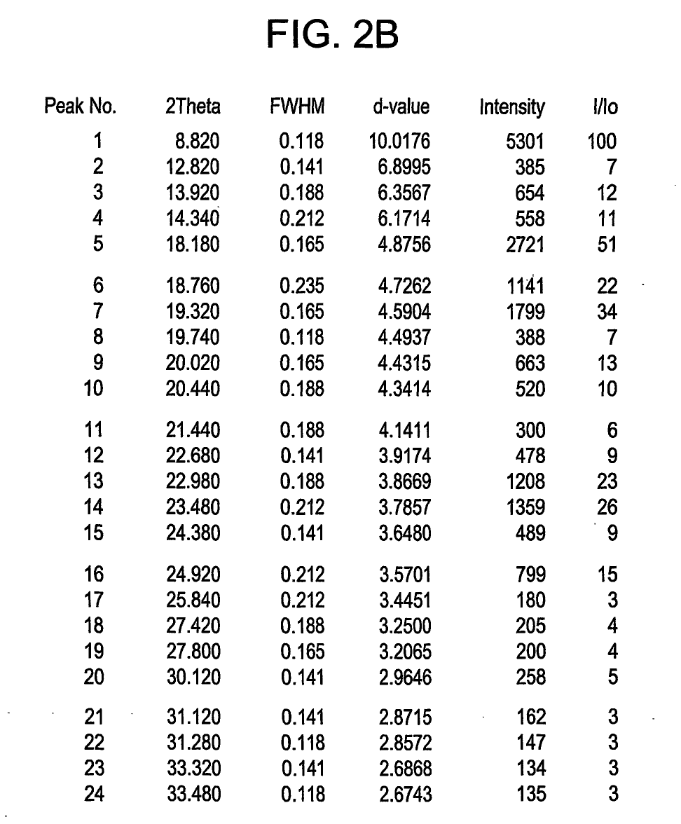

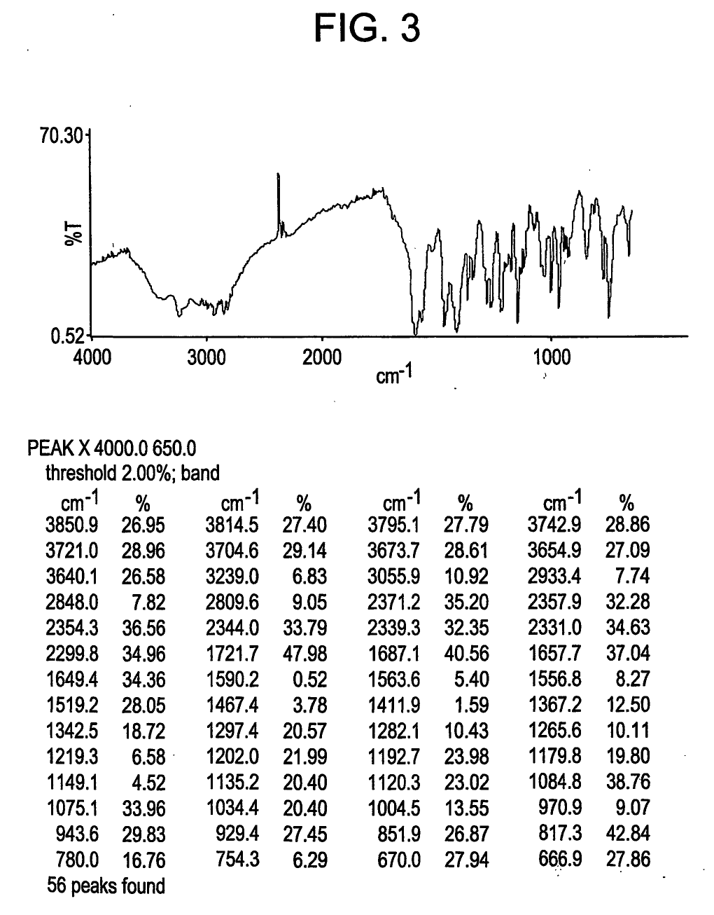

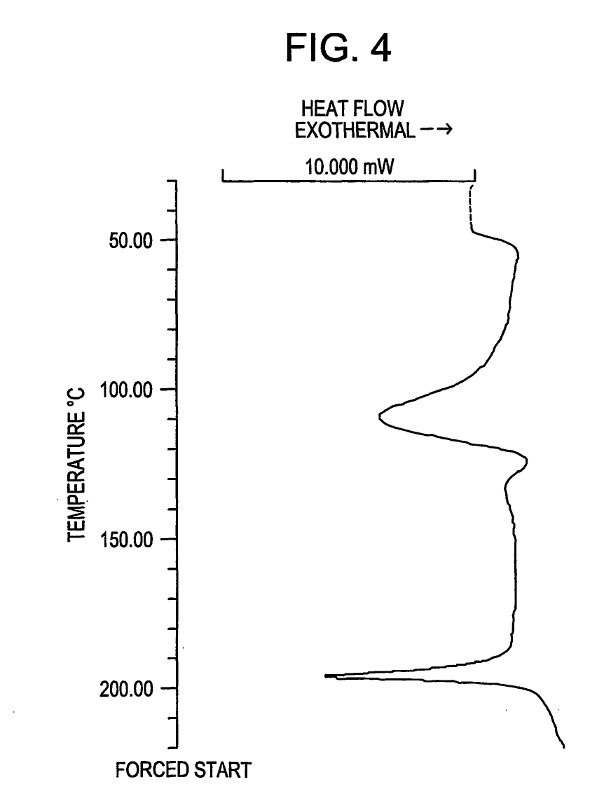

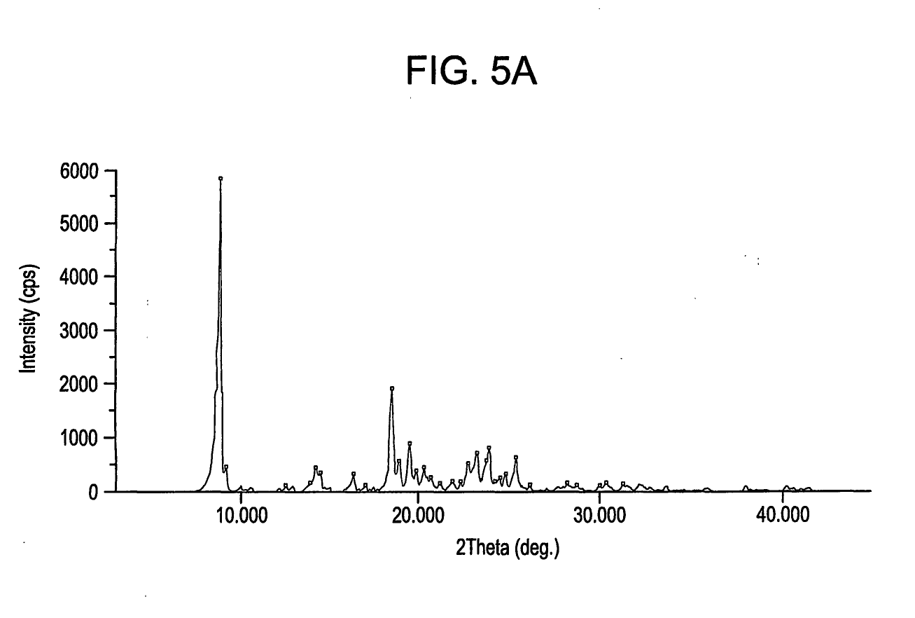

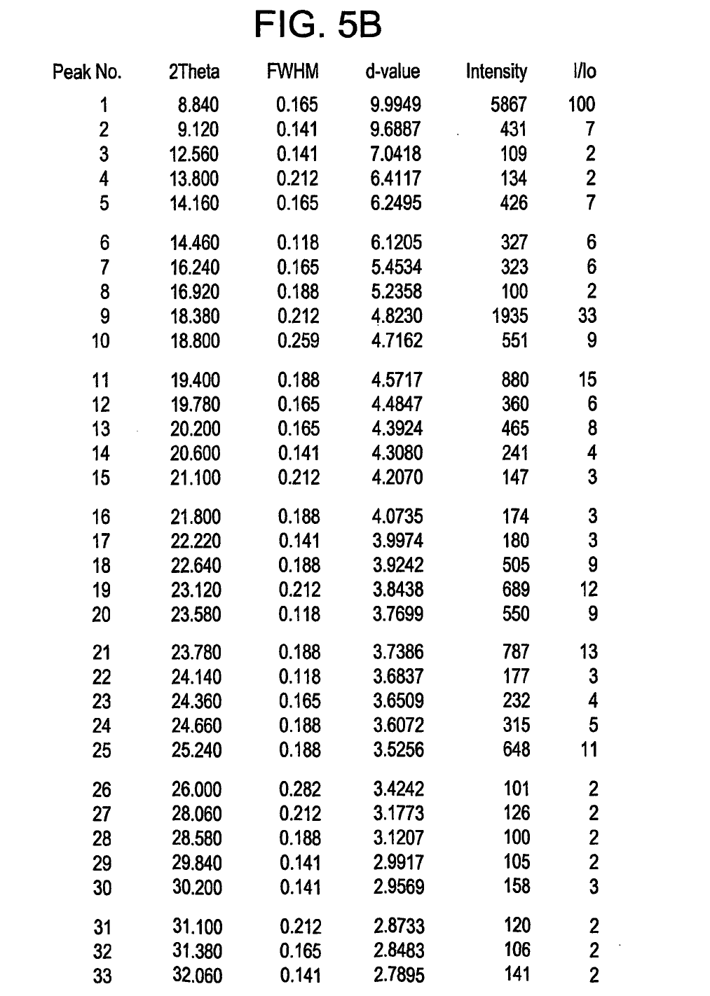

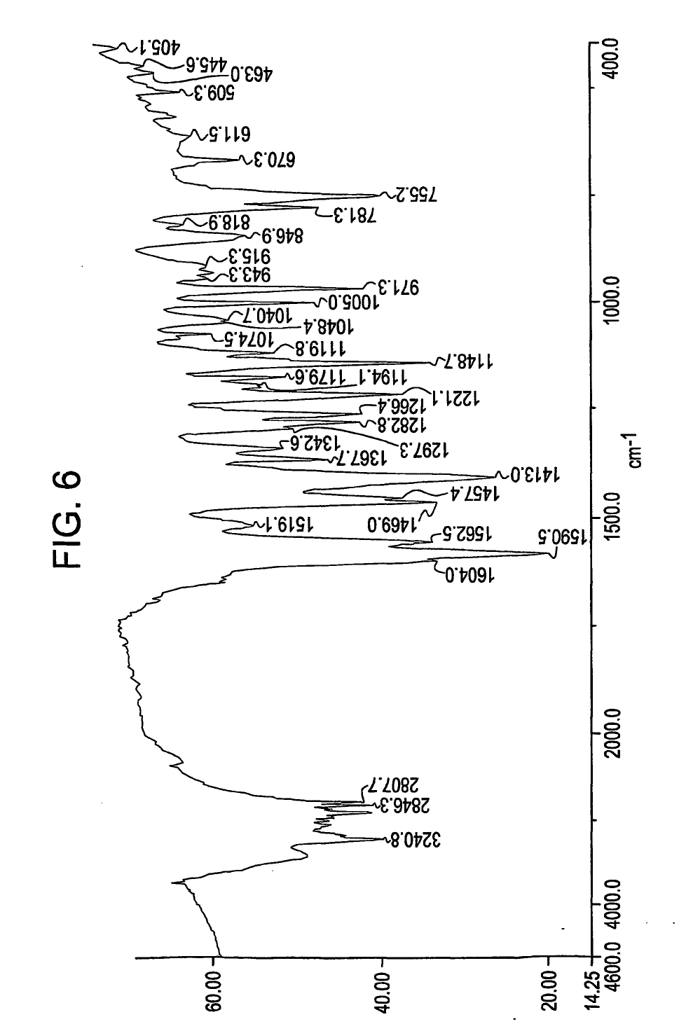

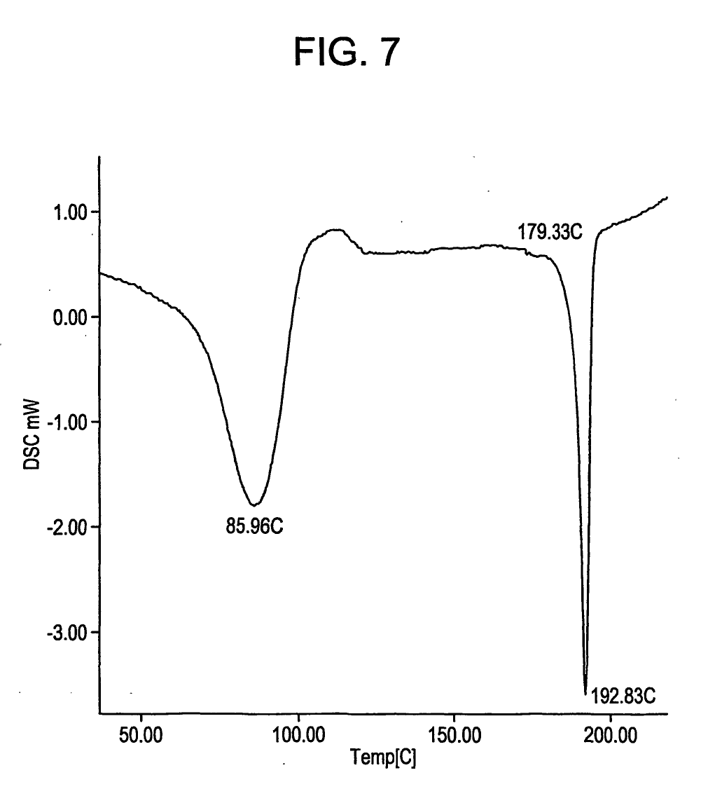

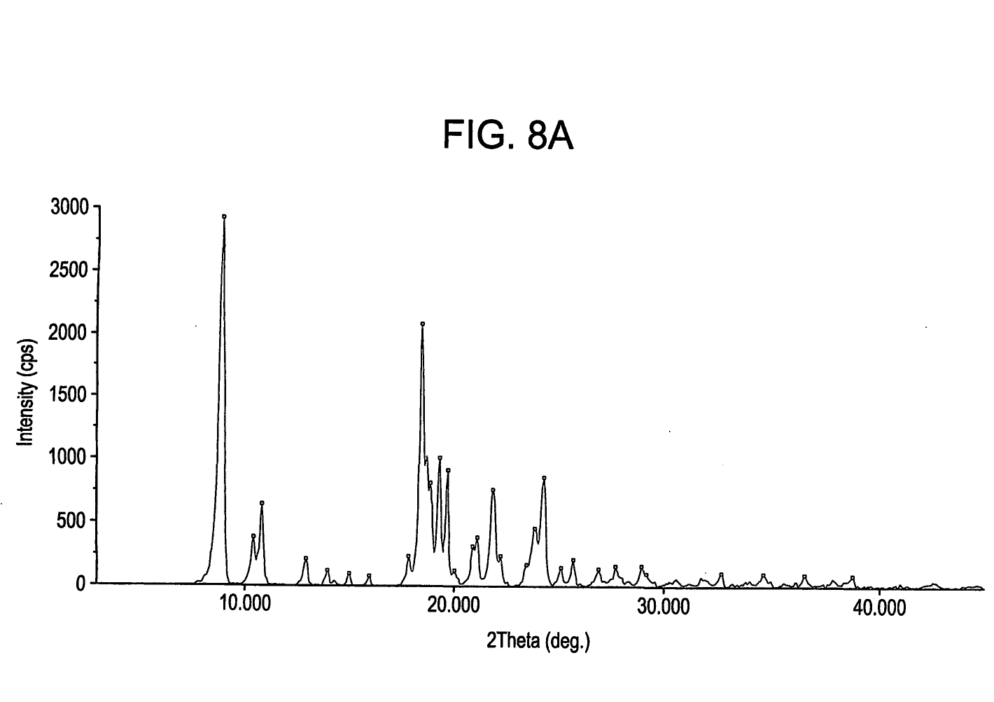

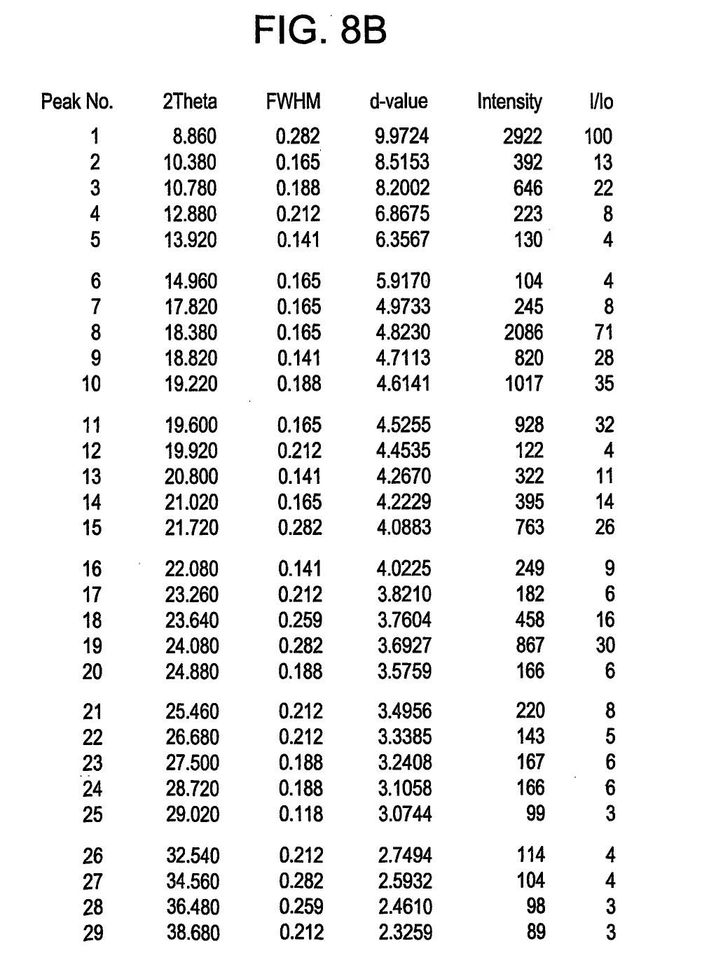

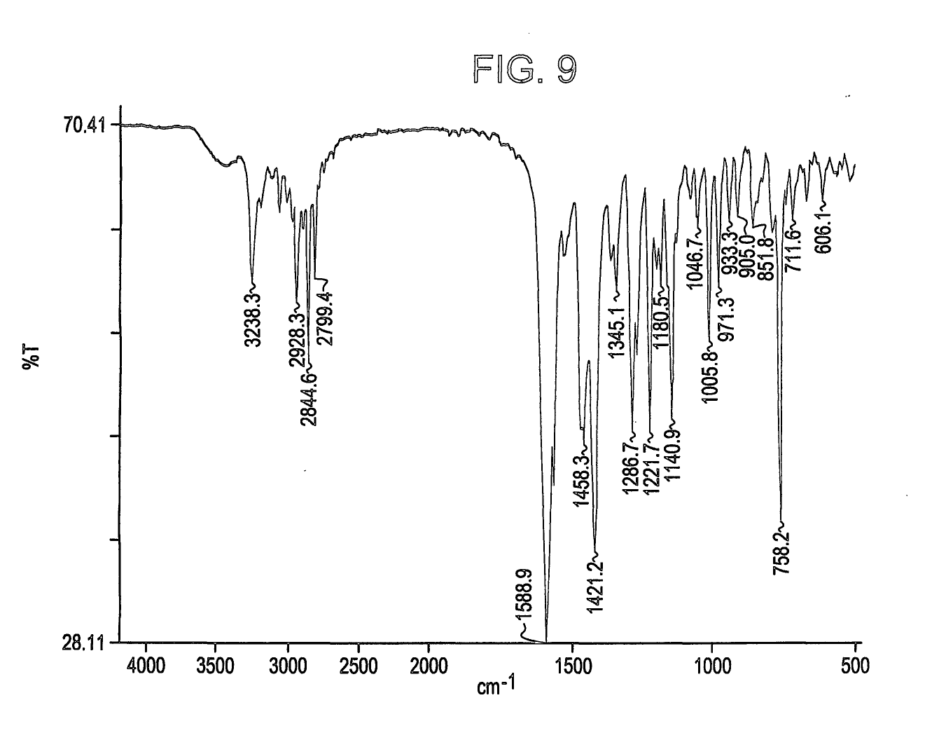

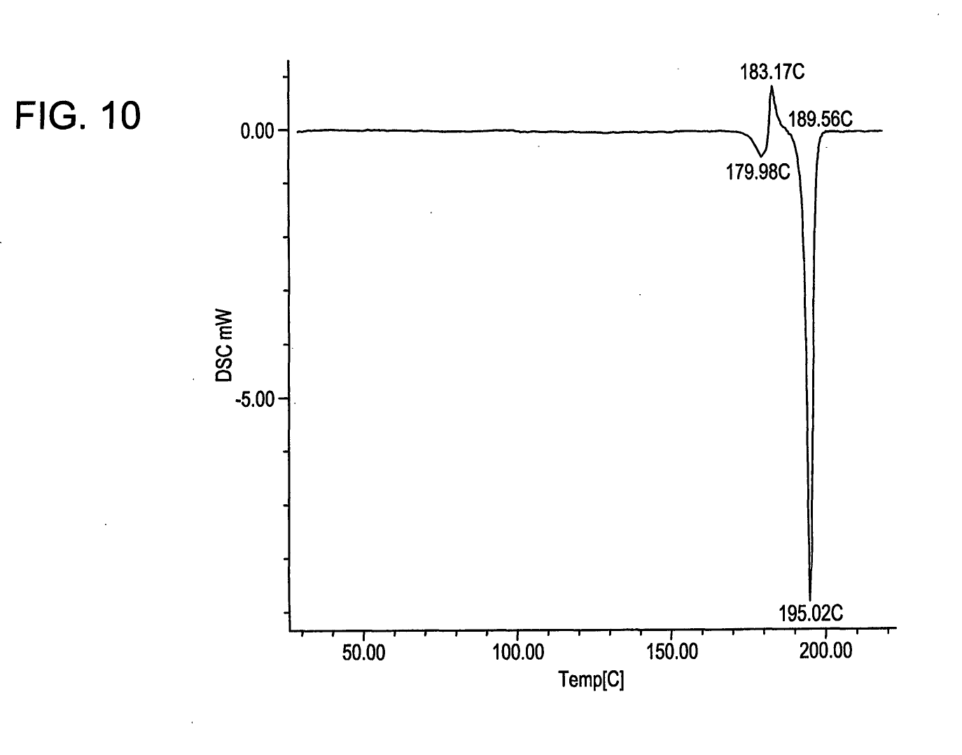

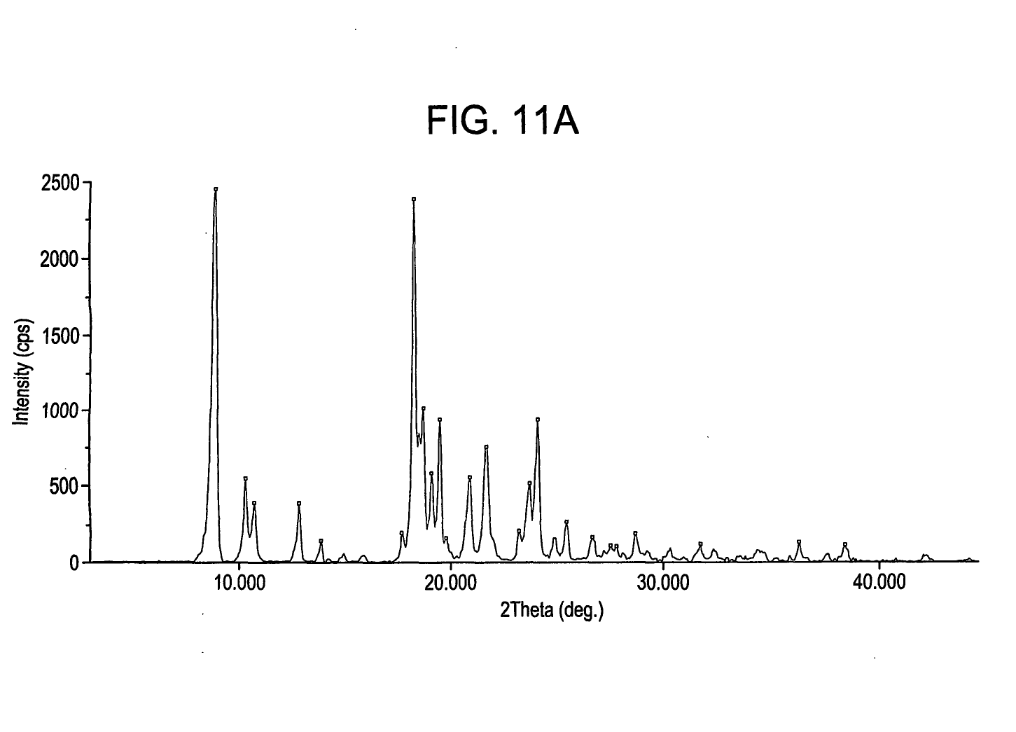

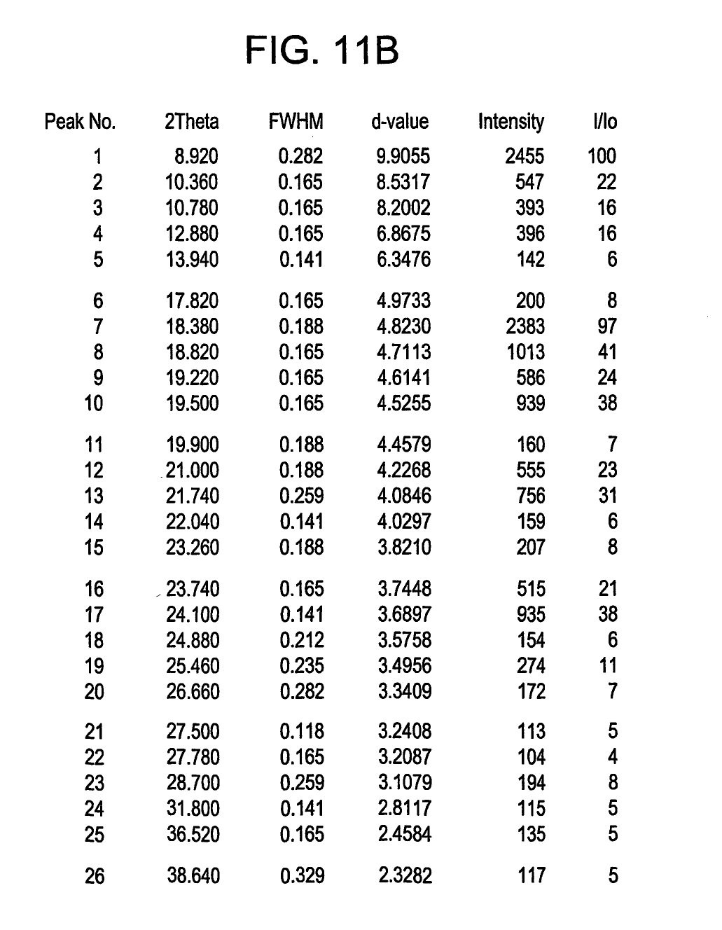

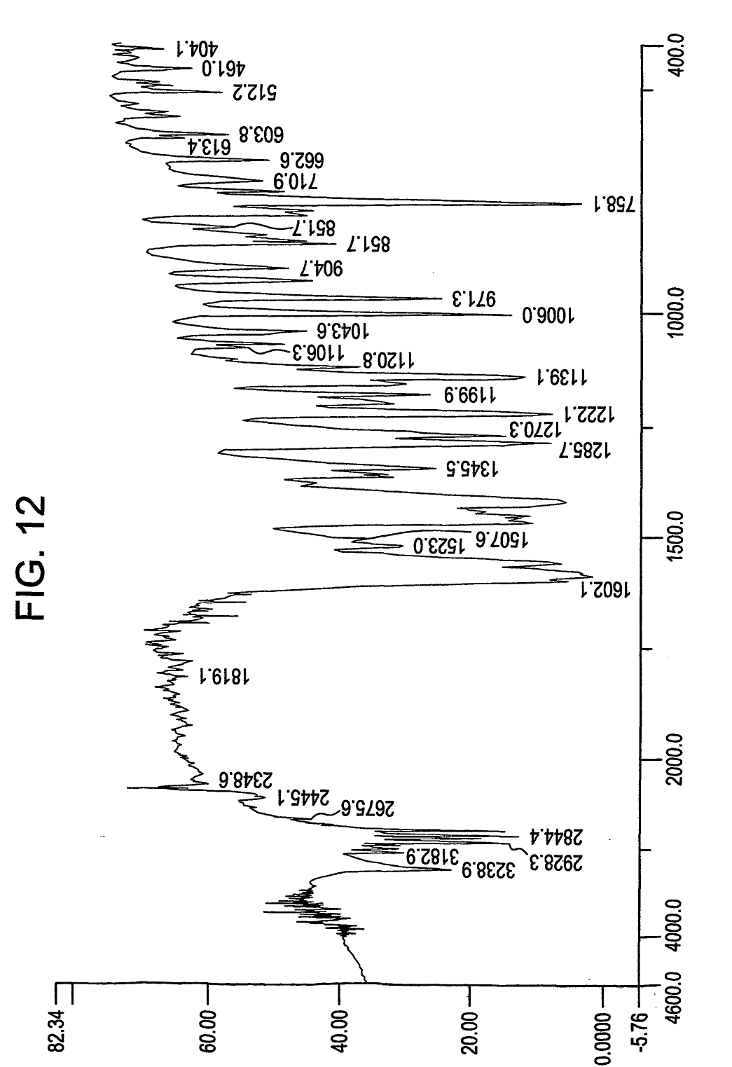

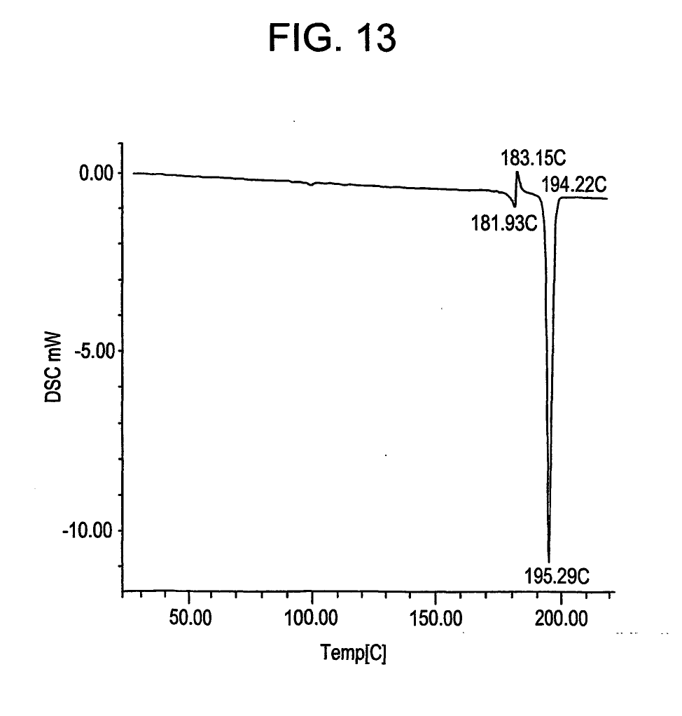

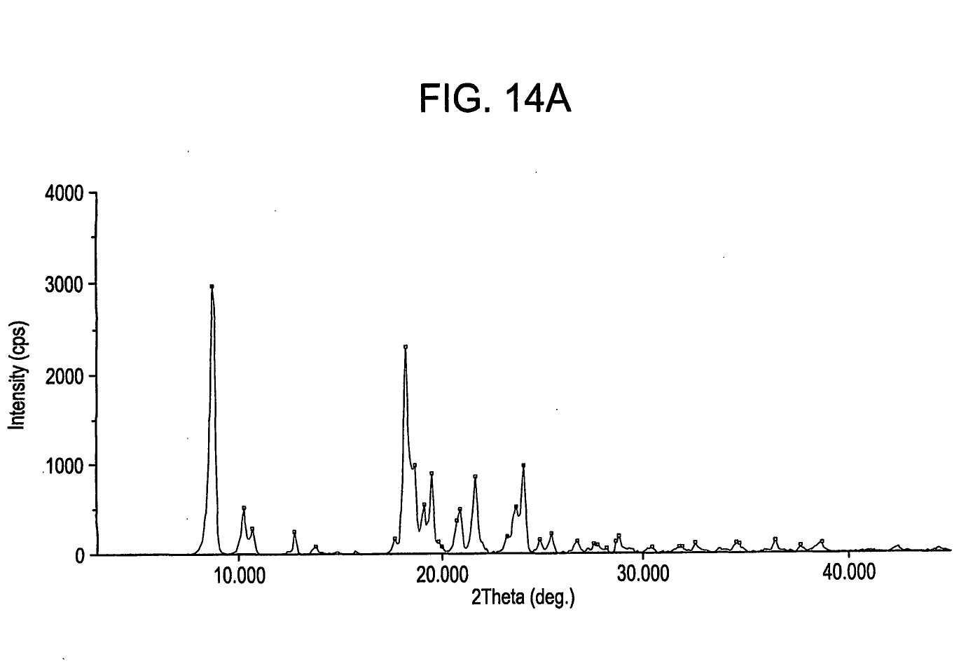

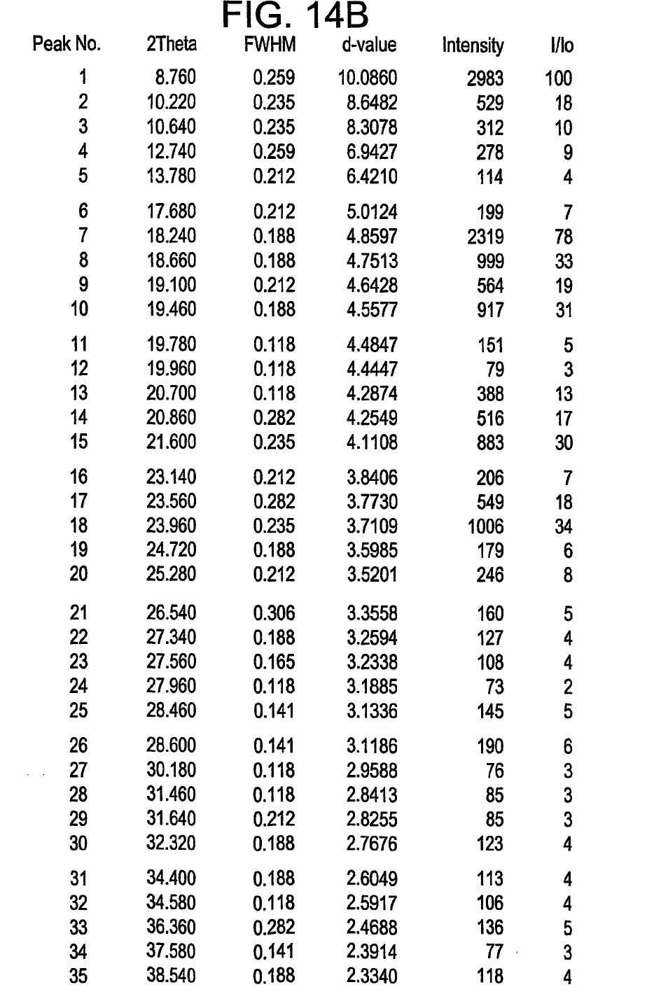

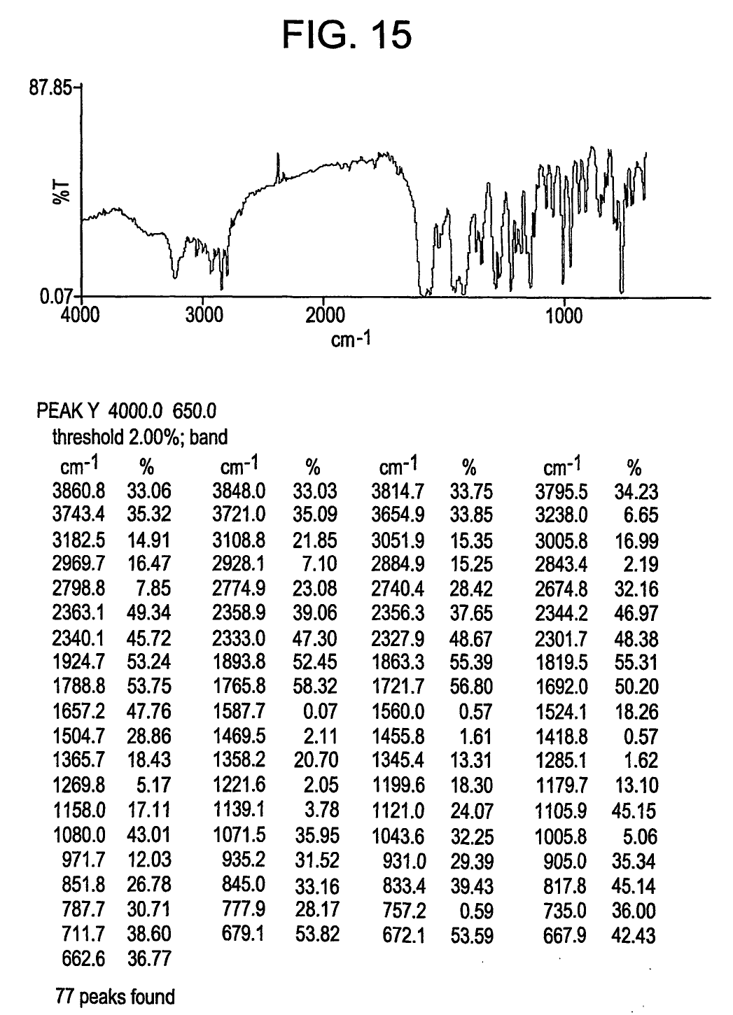

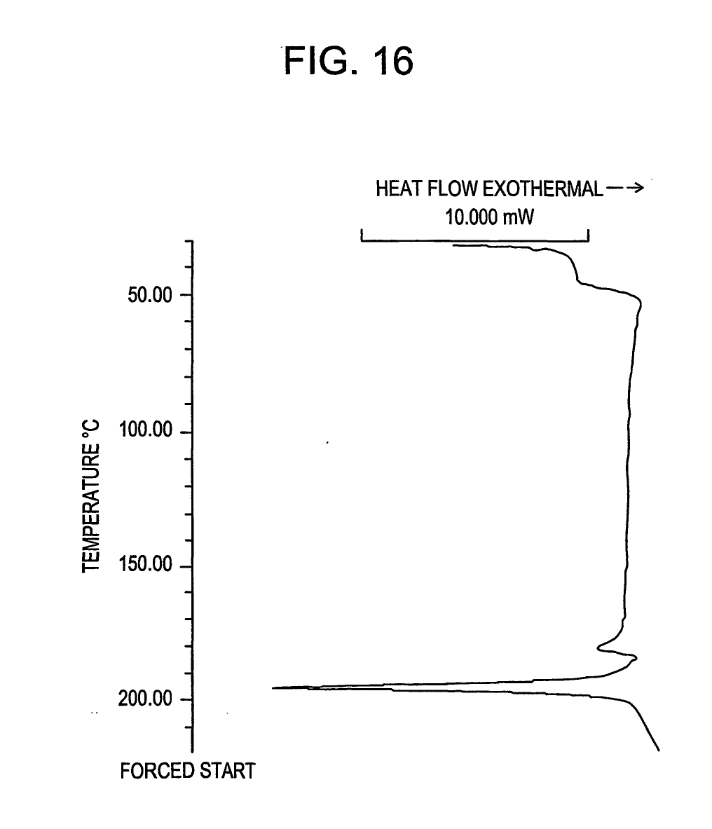

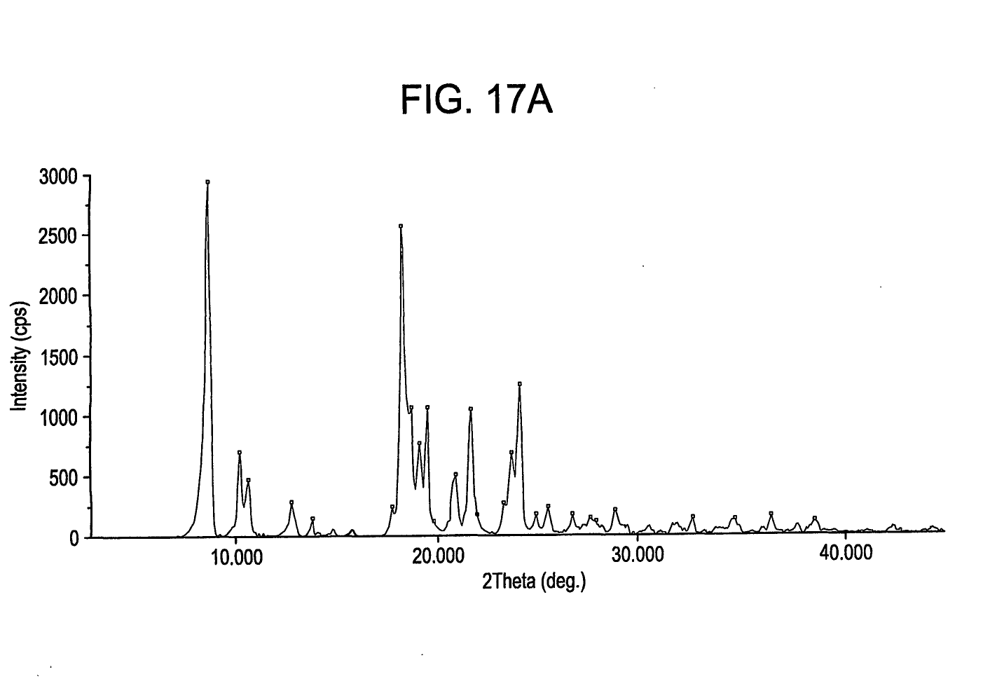

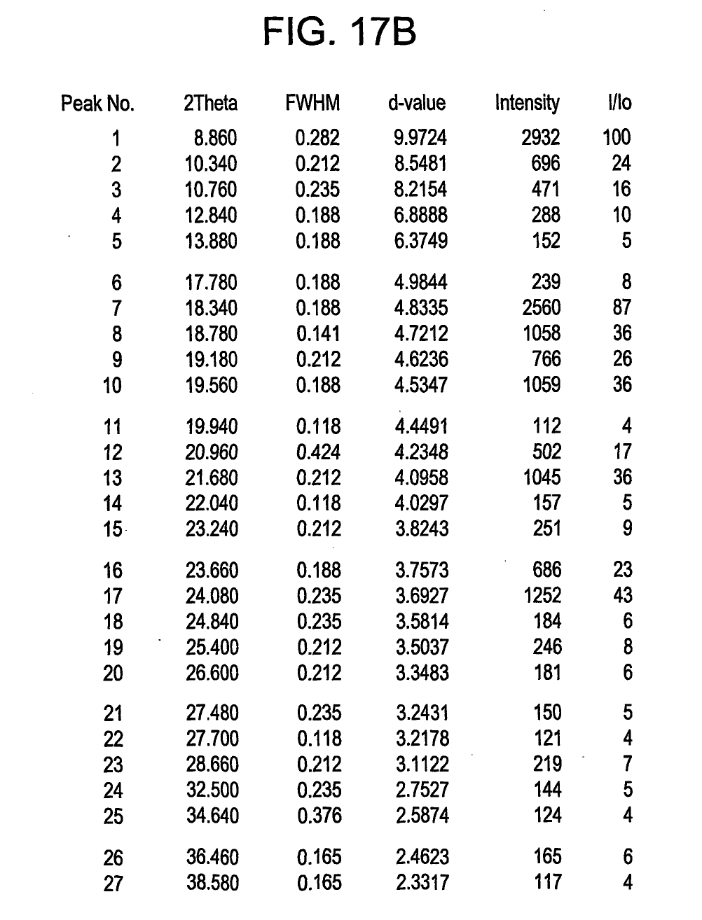

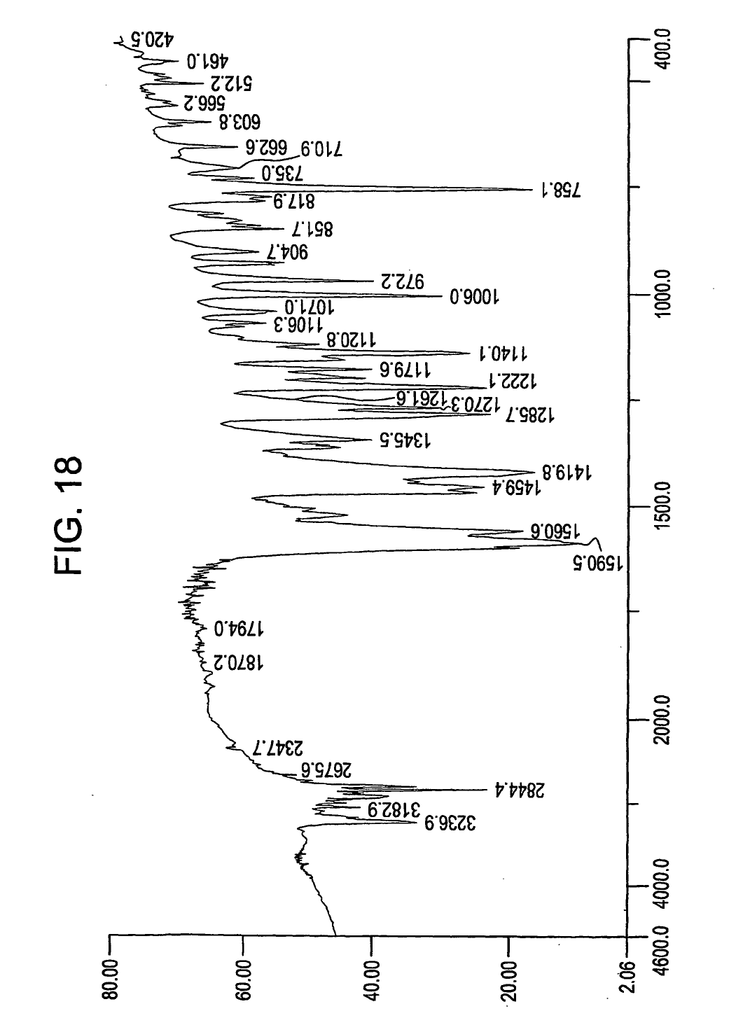

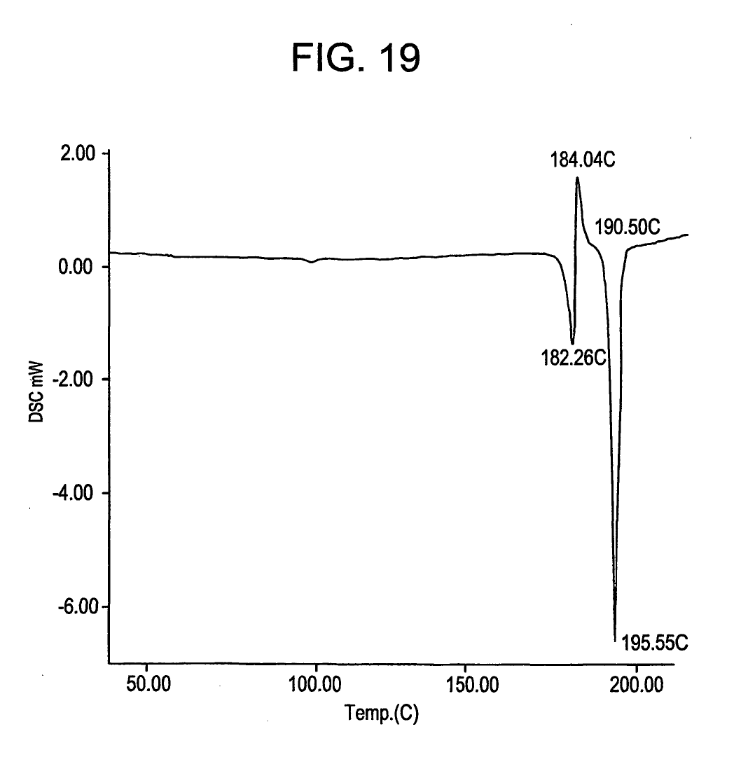

1. A compound which is Olanzapine monohydrate-I.

2. A compound which is Olanzapine dihydrate-I.

3. A compound which is Olanzapine monohydrate-I having a X-ray powder diffraction pattern as represented by the following:

| d value | I/Iₒ |
|---|---|
| 10.176 | 100 |
| 6.8995 | 7 |
| 6.3567 | 12 |
| 6.1714 | 11 |
| 4.8756 | 51 |
| 4.7262 | 22 |
| 4.5905 | 34 |
| 4.4937 | 7 |
| 4.4315 | 13 |
| 4.3414 | 10 |
| 4.1411 | 6 |
| 3.9174 | 9 |
| 3.8669 | 23 |
| 3.7857 | 26 |
| 3.6480 | 9 |
| 3.5701 | 15 |
| 3.4451 | 3 |
| 3.2500 | 4 |
| 3.2065 | 4 |
| 2.9646 | 5 |
| 2.8715 | 3 |
| 2.8572 | 3 |
| 2.6868 | 3 |
| 2.6743 | 3 |

4. A compound which is Olanzapine dihydrate-I having a X-ray powder diffraction pattern as represented by the following:

| D value | I/Iₒ |
|---|---|
| 9.9949 | 100 |
| 9.6887 | 7 |
| 7.0418 | 2 |
| 6.4117 | 2 |
| 6.2495 | 7 |
| 6.1205 | 6 |
| 5.4534 | 6 |
| 5.2358 | 2 |
| 4.8230 | 33 |
| 4.7162 | 9 |
| 4.5717 | 15 |
| 4.4847 | 6 |
| 4.3924 | 8 |
| 4.3080 | 4 |
| 4.2070 | 3 |
| 4.0735 | 3 |
| 3.9974 | 3 |
| 3.9242 | 9 |
| 3.8438 | 12 |
| 3.7699 | 9 |
| 3.7386 | 13 |
| 3.6837 | 3 |
| 3.6509 | 4 |
| 3.6072 | 5 |
| 3.5256 | 11 |
| 3.4242 | 2 |
| 3.1773 | 2 |
| 3.1207 | 2 |
| 2.9917 | 2 |
| 2.9569 | 3 |
| 2.8733 | 2 |
| 2.8483 | 2 |
| 2.7895 | 2 |

5. A process for preparing olanzapine monohydrate-I which comprises the steps of:
a) refluxing a mixture of 4-amino-2-methyl-10H-thieno-[2,3-b][1,5]benzodiazepine hydrochloride, N-methyl piperazine, dimethyl sulfoxide and toluene for 5 to 20 hours;
b) cooling the mixture to 20 to 90° C.;
c) adding water;
d) cooling the mixture to −5 to 25° C. and stirring for 2-10 hours;
e) filtering the mixture and washing with water; and
f) drying at 30 to 50° C. to a constant weight.

6. The process according to claim 5, wherein the amounts of 4-amino-2-methyl-10H-thieno-[2,3-b][1,5]benzodiazepine hydrochloride and N-methyl piperzine are in the ratio of 1:2.0-8.4.

7. The process according to claim 5, wherein the volume of dimethyl sulfoxide is 2-8 times the number of moles of 4-amino-2-methyl-10H-thieno-[2,3-b][1,5]benzodiazepine hydrochloride.

8. The process according to claim 5, wherein the volume of toluene is 3-8 times the number of moles of 4-amino-2-methyl-10H-thieno-[2,3-b][1,5] benzodiazepine hydrochoride and dimethyl sulfoxide.

9. A process for preparing olanzapine dihydrate-I which comprises the steps of:
a) refluxing a mixture of 4-amino-2-methyl-10H-thieno-[2,3-b][1,5] benzodiazepine hydrochloride, N-methyl piperazine, dimethyl sulfoxide and toluene for 5 to 20 hours;
b) cooling the mixture to 20 to 90° C.;
c) adding water;
d) cooling the mixture to −5 to 25° C. and stirring for 2-10 hours;
e) filtering the mixture and washing with water; and
drying at ambient temperature to a constant weight.

10. The process according to claim 9, wherein the amounts of 4-amino-2-methyl-10H-thieno-[2,3-b][1,5-benzodiazepine hydrochloride and N-methyl piperzine are in the ratio of 1:2.0-8.4.

11. The process according to claim 9, wherein the volume of dimethyl sulfoxide is 2-8 times the number of moles of 4-amino-2-methyl-10H-thieno-[2,3-b[1,5] benzodiazepine hydrochloride.

12. The process according to claim 9, wherein the volume of toluene is 3-8 times the number of moles of 4-amino-2-methyl-10H-thieno-[2,3-b][1,5] benzodiazepine hydrochloride.

13. A process for preparing Olanzapine Form-1 from Olanzapine dihydrate-I which comprises the steps of:
a) stirring 2-methyl-4-(4-methyl-1-piperazinyl)-10H-thieno-[2,3-b][1,5] benzodiazepine (olanzapine monohydrate-I) in dichloromethane at reflux to obtain a clear solution;
b) treating the solution with carbon;
c) filtering the solution to obtain a filtrate;
d) cooling the filtrate to 0 to 5° C.
e) stirring for 60-90 minutes;
f) filtering to obtain a solid, washing and drying at 60° to 70° C. to a constant weight.

14. The process according to claim 13, wherein in step f) the solid is washed with dichloromethane.

15. The process according to claim 13, wherein the amount of dichloromethane used in step a) is 4.5 to 13 volume/weight of 2-methyl-10H-thieno[2,3-b][1,5] benzodiazepine hydrochloride.

16. A process for preparing olanzapine Form-1 from olanzapine monohydrate-I which comprises the steps of:
a) stirring 2-methyl4-(4-methyl-1-piperazinyl)-10H-thieno-[2,3-b][1,5]benzodiazepine in dichloromethane at reflux to obtain a clear solution;
b) treating the solution with carbon;
c) filtering the solution to obtain a filtrate;
d) cooling the filtrate to below 0 to 5° C.; and
e) stirring for 60-90 minutes, f) separating the solid, washing and drying at 60° to 70° C. to a constant weight.

17. The process according to claim 16, wherein the amount of dichloromethane used is 4.5 to 13 volume/weight of 2-methyl-4-(4-methyl-1-piperazinyl)-10H-thieno-[2,3-b][1,5]benzodiazepine hydrochloride.

18. The process according to claim 16, wherein is step f) the solid is washed with dichloromethane.

19. A process for preparing Olanzapine Form-I from Olanzapine Form-2 which comprises the steps of:
a) stirring 2-methyl-10H-thieno-[2,3-b][1,5]benzodiazepine hydrochloride (olazapine Form-2) in dichloromethane at reflux to obtain a clear solution;
b) filtering and cooling the filtrate to 0 to 5° C.;
c) stirring for 60-90 minutes; and
d) filtering to obtain a solid, washing and drying at 60-70° C. to a constant weight.

20. The process according to claim 19, wherein the amount of dichloromethane used is 4.5 to 13 volume/weight of 2-methyl-10H-thieno-[2,3-b][1,5]benzodiazepine hydrochloride.

21. The process according to claim 19, wherein in step d) the solid is washed with dichloromethane.
